(19) United States
(12) Patent Application Publication
(10) Pub. No.: US 2013/0310571 A1
(43) Pub. Date: Nov. 21, 2013

(54) METHODS FOR THE PREPARATION OF BENDAMUSTINE

(71) Applicants: Anton H. Gayring, Holstein (CH); Scott A. Miller, Drexel Hill, PA (US)

(72) Inventors: Anton H. Gayring, Holstein (CH); Scott A. Miller, Drexel Hill, PA (US)

(73) Assignee: Cephalon, Inc, Frazer, PA (US)

(21) Appl. No.: 13/981,480

(22) PCT Filed: Jan. 18, 2012
(86) PCT No.: PCT/US12/21686

§ 371 Date: Jul. 24, 2013

Related U.S. Application Data
(60) Provisional application No. 61/437,809, filed on Jan. 31, 2011.

(51) Int. Cl. C07D 235/16

(52) U.S. Cl. CPC C07D 235/16
USPC 548/310.1

(57) ABSTRACT

Improved methods for the preparation and purification of bendamustine hydrochloride are described; such as method of preparing bendamustine hydrochloride comprising contacting a compound of formula HBI: with thionyl chloride

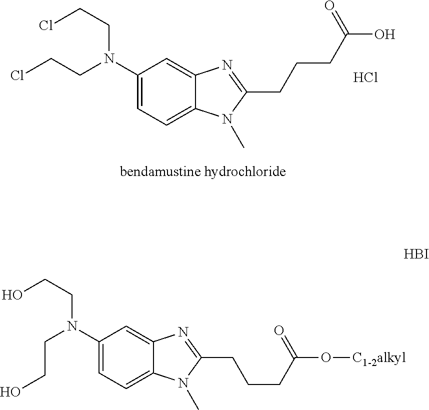

### TECHNICAL FIELD

The invention is directed to methods of preparing bendamustine hydrochloride.

### BACKGROUND

Bendamustine hydrochloride, i.e., the hydrochloride salt of 4-{5-[Bis(2-chloroethyl)amino]-1-methyl-2-benzimidazolyl}butyric acid:

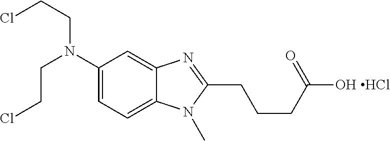 [SMILES: Cn1c(CCCC(=O)O)nc2cc(N(CCCl)CCCl)ccc21 ; Cl]

Bendamustine Hydrochloride was initially synthesized in 1963 in the German Democratic Republic (GDR) and was available from 1971 to 1992 there under the tradename Cytostasan®. See, e.g., W. Ozegowski and D. Krebs, IMET 3393 γ-[1-methyl-5-bis-(β-chloroethyl)-aminobenzimidazolo-(2)]-butyryl chloride, a new cytostatic agent of the group of benzimidazole nitrogen mustards. Zbl. Pharm. 110, (1971) Heft 10, 1013-1019, describing the synthesis of bendamustine hydrochloride monohydrate. Since that time, it has been marketed in Germany under the tradename Ribomustin® and is currently marketed in the United States under the tradename Treanda®. Bendamustine is an alkylating agent that has been shown to have therapeutic utility in treating diseases such as chronic lymphocytic leukemia, Hodgkin's disease, non-Hodgkin's lymphoma, multiple myeloma, and breast cancer.

Reported commercial syntheses of bendamustine hydrochloride require as many as nine or more steps and several recrystallizations. During synthesis, it is desirable to keep the impurity profile of the bendamustine hydrochloride within acceptable limits. Methods of improving the synthesis of bendamustine hydrochloride are needed.

### SUMMARY

Methods of making bendamustine hydrochloride are described. The methods of the invention comprise:

(a) contacting a compound of formula HBI:

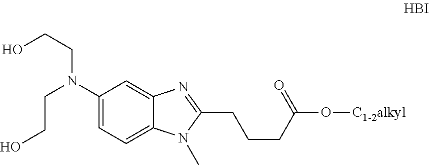

with thionyl chloride in the presence of an organic solvent at about 25° C. for at least about 20 hours to form a first product solution;
(b) combining the first product solution with an aqueous solution of hydrochloric acid to form a biphasic mixture;
(c) removing the organic phase from the biphasic mixture to provide a first aqueous mixture;
(d) heating the first aqueous mixture for 4 to 5 hours at 85° C. to 90° C.;
(e) distilling the first aqueous mixture at between 50° C. to 60° C. to remove between 65-75% of the aqueous solution of hydrochloric acid from the first aqueous mixture to provide a second aqueous mixture;
(f) crystallizing a first portion of bendamustine hydrochloride from the second aqueous mixture;
(g) isolating the first portion of bendamustine hydrochloride from the second aqueous mixture;
(h) heating the first portion of bendamustine hydrochloride in acetone to provide a second portion of bendamustine hydrochloride; and
(i) isolating the second portion of bendamustine hydrochloride;
wherein the methods do not include a step comprising recrystallization of bendamustine hydrochloride using ethanol.

Also within the scope of the invention are methods of purifying bendamustine hydrochloride. These methods comprise (a) heating bendamustine hydrochloride in an aqueous solution of hydrochloric acid to form a mixture; (b) distilling off at least part of the aqueous solution of hydrochloric acid from the mixture; (c) adding water to form a bendamustine solution; (d) crystallizing bendamustine hydrochloride from the bendamustine solution; (e) isolating the bendamustine hydrochloride from the bendamustine solution; (f) heating the isolated bendamustine hydrochloride in acetone.

### DETAILED DESCRIPTION OF ILLUSTRATIVE EMBODIMENTS

A present commercial preparation of bendamustine hydrochloride includes the conversion of HBI ethylbutyrate to bendamustine using thionyl chloride followed by heating in the presence of hydrochloric acid:

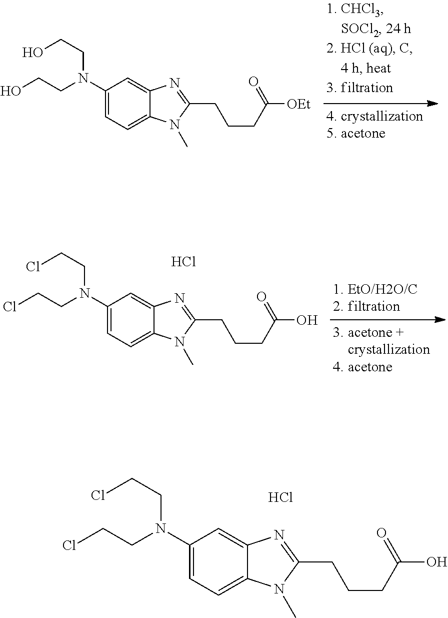 [SMILES: CCOC(=O)CCCc1nc2cc(N(CCO)CCO)ccc2n1C ; Cn1c(CCCC(=O)O)nc2cc(N(CCCl)CCCl)ccc21 ; Cl ; Cn1c(CCCC(=O)O)nc2cc(N(CCCl)CCCl)ccc21 ; Cl]

The resulting product is recrystallized from ethanol to remove impurities. The ethanol recrystallization step, while adequately providing purified bendamustine hydrochloride, imposes additional costs to the total synthesis of bendamustine hydrochloride. Moreover, the presence of ethanol increases the probability that additional bendamustine ethyl ester (“BM1 EE”), an undesired, toxic side-product, will form:

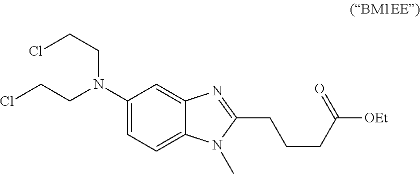 [SMILES: CCOC(=O)CCCc1nc2cc(N(CCCl)CCCl)ccc2n1C]

In addition to bendamustine ethyl ester, the level of other potential impurities must also be kept within acceptable limits. Those impurities include:

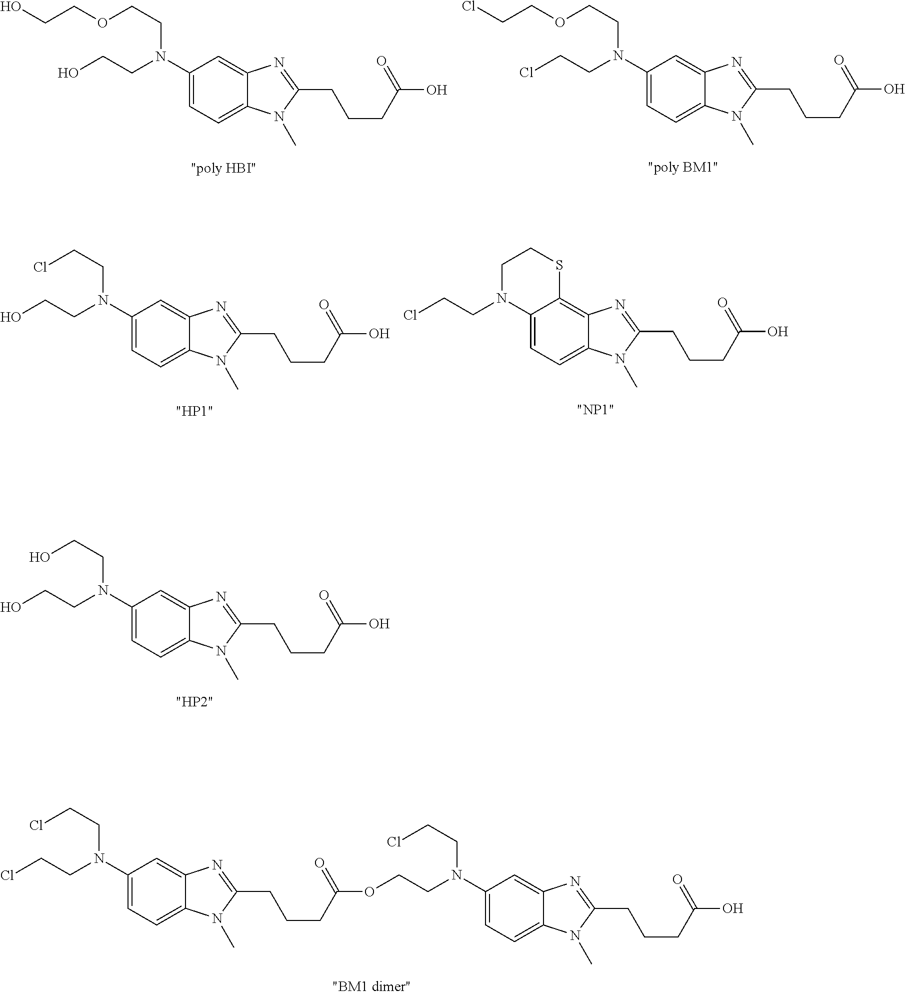

It has been discovered that bendamustine hydrochloride with an acceptable purity profile can be prepared without having to perform an additional recrystallization from ethanol. As such, the methods of the invention described herein expressly exclude any step comprising recrystallization of bendamustine hydrochloride using ethanol.

The methods of the invention generally provide for a bendamustine hydrochloride product having 0.1% or less of each of BM1EE, poly HBI, poly BM1, HP1, NP1, HP2, and BM1 dimer. Preferably, the methods of the invention generally provide for a bendamustine hydrochloride product having 0.1% or less of each of NP1, BM1 dimer, and BM1EE.

It has also been discovered that bendamustine hydrochloride can be recrystallized from aqueous hydrochloric acid, with the optional use of charcoal. These methods generally provide for a bendamustine hydrochloride product having 0.1% or less of each of BM1EE, poly HBI, poly BM1, HP1, NP1, HP2, and BM1 dimer. Preferably, the methods of the invention generally provide for a bendamustine hydrochloride product having 0.1% or less of each of BM1 dimer, BM1EE, HP1, and HP2.

According to the invention, the preparation of bendamustine hydrochloride begins by contacting a compound of formula HBI:

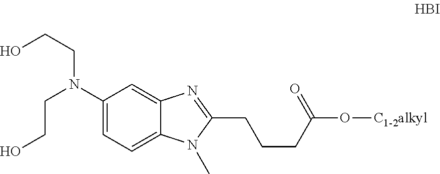

wherein C₁₋₂alkyl refers to methyl or ethyl;

with thionyl chloride (SOCl₂) in the presence of an organic solvent at about 25° C. for at least about 20 hours to form a first product solution. As used herein, “contacting” includes any method that facilitates the reaction of thionyl chloride with HBI. For example, the thionyl chloride can be added to a solution of HBI and the organic solvent and then the mixture can be stirred, shaken, agitated, or the like. One exemplary organic solvent is chloroform. It is preferable that about 2.05 to about 2.20 equivalents of thionyl chloride, based on the amount of HBI, be used, with 2.11 equivalents being most preferred. The addition of thionyl chloride is exothermic, therefore cooling, for example, about 0° C. to about 5° C., can be employed during the thionyl chloride addition, followed by warming to about 25° C. As used herein, “about 25° C.,” refers to an internal temperature of 25° C.±5° C. As those skilled in the art will appreciate, “about 25° C.,” is also consistent with ambient room temperature. The HBI should be in contact with thionyl chloride for at least about 20 hours. The contact may be as long as about 30 hours. Preferably, after the thionyl chloride is added, the HBI should be in contact with the thionyl chloride for about 20 to 24 hours.

Optionally, after the contacting step, excess thionyl chloride can be removed via degassing using inert gas, for example nitrogen or argon.

Also with the scope of the invention, the first product solution is combined with an aqueous solution of hydrochloric acid to form a biphasic mixture. As used herein, “combined” refers to any methods that bring the aqueous solution of hydrochloric acid in contact with the first product solution, resulting in the organic products being transferred from the organic phase to the aqueous phase. Preferably, the aqueous solution of hydrochloric acid is technical grade hydrochloric acid, which typically contains about 32% hydrochloric acid. Preferably, about 2.8 times the initial mass of HBI of aqueous hydrochloric acid is combined with the first product solution. Those skilled in the art will readily appreciate that the amount of aqueous solution of hydrochloric acid can be readily modified based on the concentration of hydrochloric acid in the aqueous solution of hydrochloric acid.

Also within the scope of the invention, the organic phase is removed from the biphasic mixture to provide a first aqueous mixture. As used herein, “removed” refers to the process of allowing the organic phase and the aqueous phase to form two layers followed by substantially all of the organic phase being separated from the aqueous phase. It is preferable that as much of the organic phase as possible, within the technical limitations of the reaction apparatus and the skill of the operating personnel, is separated from the aqueous phase. Any conventional means of separating the organic phase from the aqueous phase are envisioned as within the scope of the invention. After its removal, the organic phase can be discarded in accordance with industry guidance.

Optionally, charcoal, preferably medicinal grade or activated charcoal, can be added to the first aqueous mixture. If charcoal is added, about 0.02 times the mass of HBI of charcoal is added to the first aqueous mixture. The first aqueous mixture, optionally containing charcoal, is heated to an internal refluxing temperature, preferably an internal temperature of about 80° C. to 90° C., preferably 85° C. to 90° C. for about 3 to 6 hours, preferably 4 to 5 hours. The step converts at least a portion of C₁₋₂alkyl ester to carboxylic acid. After heating, if charcoal was added, the charcoal can be removed, preferably via filtration. Aqueous hydrochloric acid solution can be used for rinsing.

Within the scope of the invention, the first aqueous mixture is distilled to remove at least a portion of the aqueous solution of hydrochloric acid to provide a second aqueous mixture. The distillation step further converts C₁₋₂alkyl ester to carboxylic acid. It has been identified that the time and temperature of the distillation not only assists in converting C₁₋₆alkyl ester to carboxylic acid, but if time and temperature are properly regulated during the distillation step, formation of BM1 dimer can be minimized. It has been determined that an internal temperature of less than or equal to 65° C., preferably 50 to 60° C., is advantageous for the distillation step. It is also preferred that the distillation be performed under reduced pressure. Ideally, the pressure should be the pressure at which the first aqueous mixture achieves reflux when the internal temperature is less than or equal to 65° C., preferably 50 to 60° C. Those skilled in the art can readily identify such pressures using routine experimentation.

It has also been observed that the amount of aqueous hydrochloric acid solution that is removed impacts the amount of BM1 dimer formation. Removal of less than or equal to 82%, preferably about 65 to 75%, of the total amount of aqueous hydrochloric acid solution minimizes the formation of BM1 dimer.

Also within the scope of the invention, a first portion of bendamustine hydrochloride is crystallized from the second aqueous mixture. The addition of warm water can assist in the crystallization. Preferably, about 4 times the mass of HBI of warm water can be added. The second aqueous solution can be seeded with crystals of bendamustine hydrochloride to assist in crystallization of bendamustine hydrochloride from the second aqueous mixture. After bendamustine hydrochloride crystallizes from the second aqueous mixture, the resulting suspension can be cooled, preferably to about 15 to 25° C. for 1 to 2 hours or overnight.

Within the scope of the invention, the first portion of bendamustine hydrochloride is isolated from the second aqueous mixture, preferably via filtration, though other methods of isolation, for example, decanting, can be employed. The isolated bendamustine hydrochloride can be washed with water and acetone. Preferably, the water and acetone used for washing is cold, for example about 0 to about 5° C. Preferably, the isolated bendamustine hydrochloride is washed at least once with water, preferably three times with water. It is preferred that the isolated bendamustine hydrochloride is washed at least once with acetone, preferably at least three times with acetone.

Also within the scope of the invention, the first portion of bendamustine hydrochloride is heated in acetone to provide a second portion of bendamustine hydrochloride. It has been observed that heating the first portion of bendamustine hydrochloride in acetone assists in the removal of bendamustine ethyl ester. Preferably, the first portion of bendamustine hydrochloride is heated in refluxing acetone. It is desirable that the heating occur for at least one hour. The heating step can be performed at least three times, preferably four times.

Within the scope of the invention, the second portion of bendamustine hydrochloride is isolated. Preferably, the isolation is via filtration, though other methods of isolation, as previously described, are also within the scope of the invention.

As previously noted, none of the steps of the methods of the inventions includes a step wherein bendamustine hydrochloride is recrystallized from ethanol.

It may be desirable to further purify the second portion of bendamustine hydrochloride to remove impurities such as BM1 dimer, BM1EE, HP1 and HP2. Charcoal can also be used in order to remove colored impurities. As such, also within the scope of the invention are methods wherein the second portion of bendamustine hydrochloride is dissolved in an aqueous solution of hydrochloric acid to form a third aqueous mixture. As used herein, “dissolved” encompasses embodiments wherein at least 90% of the second portion of bendamustine hydrochloride, preferably at least 95%, most preferably at least 99% of the bendamustine hydrochloride dissolves in the aqueous solution of hydrochloric acid. Preferably, about two times the mass of the second portion of bendamustine hydrochloric acid of the aqueous solution of hydrochloric acid is added. The aqueous solution of hydrochloric acid is preferably technical grade, i.e., about 32% of hydrochloric acid in water, though other concentrations are within the scope of the invention.

Within the scope of the invention, the third aqueous mixture is heated to an internal refluxing temperature, preferably an internal temperature of about 80° C. to 90° C., preferably 85° C. to 90° C. for about 3 to 6 hours, preferably 4 to 5 hours. Optionally, charcoal can be added to the third aqueous mixture. Preferably, about 0.05 times the amount of the second portion of bendamustine hydrochloride of charcoal is added.

Also within the scope of the invention, the third aqueous mixture is distilled to remove at least a portion of the aqueous hydrochloric acid solution to form a fourth aqueous mixture. If charcoal has been added to the third aqueous mixture, the charcoal is preferably removed, for example by filtration, prior to distillation. It has been determined that an internal temperature of less than or equal to 65° C., preferably 50 to 60° C., is advantageous for the distillation step. It is also preferred that the distillation be performed under reduced pressure. Ideally, the pressure should be the pressure at which the first aqueous mixture achieves reflux when the internal temperature is less than or equal to 65° C., preferably 50 to 60° C. Those skilled in the art can readily identify such pressures using routine experimentation. Preferably, 0.5 to 1 times the weight of the second portion of bendamustine hydrochloride of the aqueous solution of hydrochloric acid is removed.

Within the scope of the invention, a third portion of bendamustine hydrochloride is crystallized from the fourth aqueous mixture. The addition of water, preferably warm water, to the fourth aqueous mixture can assist in crystallization. Also, the fourth aqueous mixture can be seeded with crystals of bendamustine hydrochloride to assist crystallization of the third portion of bendamustine hydrochloride from the fourth aqueous mixture. After the third portion of bendamustine hydrochloride crystallizes from the fourth aqueous mixture, the resulting suspension can be warmed to between 35 and 45° C. for up to 2 hours then cooled, preferably to about 15 to 25° C. for 1 to 2 hours.

Within the scope of the invention, the third portion of bendamustine hydrochloride is isolated from the fourth aqueous mixture, preferably via filtration, though other methods of isolation, for example, decanting, can be employed. The isolated bendamustine hydrochloride can be washed with water and acetone. Preferably, the water and acetone used for washing is cold, for example about 0 to about 5° C. Preferably, the isolated bendamustine hydrochloride is washed at least once with water, preferably three times with water. It is preferred that the isolated bendamustine hydrochloride is washed at least once with acetone, preferably at least three times with acetone.

Also within the scope of the invention, the third portion of bendamustine hydrochloride is heated in acetone to provide a fourth portion of bendamustine hydrochloride. It has been observed that heating the third portion of bendamustine hydrochloride in acetone assists in the removal of bendamustine ethyl ester. Preferably the third portion of bendamustine hydrochloride is heated in refluxing acetone. It is desirable that the heating occur for at least one hour. The heating step can be performed at least three times, preferably four times.

Within the scope of the invention, the fourth portion of bendamustine hydrochloride is isolated. Preferably, the isolation is via filtration, though other methods of isolation, as previously described, are also within the scope of the invention.

Also within the scope of the invention are methods of purifying bendamustine hydrochloride. These methods can be used on bendamustine hydrochloride that has been prepared according to the methods described herein or using any methods known in the art. As such, methods of the invention include heating bendamustine hydrochloride in an aqueous solution of hydrochloric acid to form a mixture. Preferably, about two times the mass of the bendamustine hydrochloride of the aqueous solution of hydrochloric acid is added. The aqueous solution of hydrochloric acid is preferably technical grade, i.e., about 32% of hydrochloric acid in water, though other concentrations are within the scope of the invention.

Within the scope of the invention, the mixture is heated to an internal refluxing temperature, preferably an internal temperature of about 80° C. to 90° C., preferably 85° C. to 90° C. for about 3 to 6 hours, preferably 4 to 5 hours. Optionally, charcoal can be added to the mixture. Preferably, about 0.05 times the amount of the bendamustine hydrochloride of charcoal is added.

Also within the scope of the invention, the mixture is distilled to remove at least a portion of the aqueous hydrochloric acid solution. If charcoal has been added to the mixture, the charcoal is preferably removed, for example by filtration, prior to distillation. It has been determined that an internal temperature of less than or equal to 65° C., preferably 50 to 60° C., is advantageous for the distillation step. It is also preferred that the distillation be performed under reduced pressure. Ideally, the pressure should be the pressure at which the mixture achieves reflux when the internal temperature is less than or equal to 65° C., preferably 50 to 60° C. Those skilled in the art can readily identify such pressures using routine experimentation. Preferably, 0.5 to 1 times the weight of the second portion of bendamustine hydrochloride of the aqueous solution of hydrochloric acid is removed.

Within the scope of the invention, water is added after distillation to form a bendamustine solution. Preferably, the water is warm.

Within the scope of the invention, bendamustine hydrochloride is crystallized from the bendamustine solution. Crystals of bendamustine hydrochloride can be added to assist the crystallization. After the bendamustine hydrochloride crystallizes from the bendamustine solution, the resulting suspension can be warmed to between 35 and 45° C. for up to 2 hours then cooled, preferably to about 15 to 25° C. for 1 to 2 hours.

Within the scope of the invention, the bendamustine hydrochloride is isolated from the bendamustine solution, preferably via filtration, though other methods of isolation, for example, decanting, can be employed. The isolated bendamustine hydrochloride can be washed with water and acetone. Preferably, the water and acetone used for washing is cold, for example about 0 to about 5° C. Preferably, the isolated bendamustine hydrochloride is washed at least once with water, preferably three times with water. It is preferred that the isolated bendamustine hydrochloride is washed at least once with acetone, preferably at least three times with acetone.

Also within the scope of the invention, the isolated bendamustine hydrochloride is heated in acetone. Preferably the isolated bendamustine hydrochloride is heated in refluxing acetone. It is desirable that the heating occur for at least one hour. The heating step can be performed at least three times, preferably four times.

Within the scope of the invention, the isolated bendamustine hydrochloride can be filtered to provide purified bendamustine hydrochloride.

### EXAMPLES

### Preparation of Bendamustine Hydrochloride from HBI Ethylbutyrate According to the Invention

HBI-ethylbutyrate (molecular weight=349.42 g/mol) was dissolved in CHCl₃ (10×mass of HBI-ethylbutyrate). The solution was cooled to about 0-5° C. and thionyl chloride (molecular weight=118.96 g/mol, 2.11 equivalents) was added over 1-2 hours. After stirring for an additional 0.25-1 hour, the mixture was warmed to 25±5° C. and stirred for an additional 20-24 hours. Aqueous hydrochloride acid (32%, 2.8×mass of HBI-ethylbutyrate) was added and the organic phase was removed. The aqueous phase was degassed at 30° C. for 15-30 minutes at 100-200 mbar). A suspension of active charcoal (0.02×mass of HBI ethylbutyrate) in aqueous hydrochloric acid (0.2×mass of HBI ethylbutyrate) was added to the aqueous phase. The mixture was heated to 85-90° C. within 1 hour and stirred for 4-5 hours at reflux. After cooling, the suspension was filtered and rinsed with aqueous hydrochloric acid (0.2×mass of HBI ethylbutyrate). The solvent was distilled under reduced pressure at an inner temperature of 65° C. About seventy percent (±5%) of the total hydrochloric acid was distilled off. Warm (35-40° C.) water (4×mass of HBI ethylbutyrate) was added. Seeding may be necessary if no crystallization occurs within 30 minutes. After crystallization, the thick suspension is cooled to about 15-25° C. and stirred for 1-2 hours or overnight at 15-25° C. The product is filtered, washed three times with water (0-5° C., total water=4×mass of HBI ethylbutyrate) and at least three times with acetone (0-5° C., total acetone=4×mass of HBI ethylbutyrate). The washed product was treated at least 4 times with acetone (2×mass of HBI ethylbutyrate) at reflux for at least 1 hour. The hot suspension was filtered and the solid dried at 35-40° C. Yield=75±15% bendamustine hydrochloride.

Recrystallization of Bendamustine Hydrochloride from Hydrochloric Acid According to the Invention

This procedure can be used to remove at least some of impurities such as BM1 dimer, BM1EE, HP1, and HP2.

Bendamustine hydrochloride to be purified was dissolved in aqueous hydrochloric acid (2×mass of bendamustine hydrochloride) and heated to 85-90° C. for at least 4-5 hours. 50-100% of the mass of aqueous hydrochloric acid was distilled off under reduced pressure at ≦65° C. Warm water was added (4×mass of bendamustine hydrochloride). The mixture can be seeded with bendamustine hydrochloride crystals, if necessary, to crystallize the bendamustine hydrochloride. After crystallization, the suspension was stirred at 40±5° C. for 0.5 to 2 hours then cooled to 20±5° C. After stirring for an additional 1-2 hours at 20±5° C., the product was filtered off, washed three times with water (0-5° C., total amount of water is 4×mass of bendamustine hydrochloride) and at least three times with acetone (0-5° C., total amount of acetone is 4×mass of bendamustine). The washed product was treated four times with acetone (2×mass of bendamustine hydrochloride each time) at reflux for at least 1 hour. The hot suspension was then filtered and the solid dried at 40° C. Yield 80±10%.

Recrystallization of Bendamustine Hydrochloride from Hydrochloric Acid/Charcoal According to the Invention

This procedure can be used to remove at least some of impurities such as BM1 dimer, BM1EE, HP1, and HP2. Colored impurities can also be removed using this procedure.

Bendamustine hydrochloride to be purified was dissolved in aqueous hydrochloric acid (2×mass of bendamustine hydrochloride) and active charcoal (0.05-0.15×mass of bendamustine hydrochloride). The suspension was heated to 85-90° C. for at least 4-5 hours. The charcoal was filtered off and rinsed with additional aqueous hydrochloric acid (0.6×mass of bendamustine hydrochloride). 50-100% of the mass of aqueous hydrochloric acid was distilled off under reduced pressure at 65° C. Warm water was added (4×mass of bendamustine hydrochloride). The mixture can be seeded with bendamustine hydrochloride crystals, if necessary, to crystallize the bendamustine hydrochloride. After crystallization, the suspension is stirred at 40±5° C. for 0.5 to 2 hours then cooled to 20±5° C. After stirring for an additional 1-2 hours at 20±5° C., the product was filtered off, washed three times with water (0-5° C., total amount of water is 4×mass of bendamustine hydrochloride) and at least three times with acetone (0-5° C., total amount of acetone is 4×mass of bendamustine). The washed product was treated four times with acetone (2×mass of bendamustine hydrochloride each time) at reflux for at least 1 hour. The hot suspension was then filtered and the solid dried at ≦40° C. Yield 80±10%.

What is claimed:

1. A method of preparing bendamustine hydrochloride 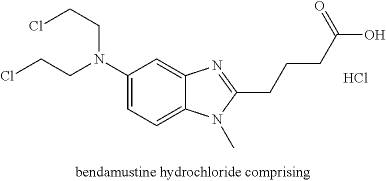 [SMILES: Cn1c(CCCC(=O)O)nc2cc(N(CCCl)CCCl)ccc21 ; Cl]
(a) contacting a compound of formula HBI: 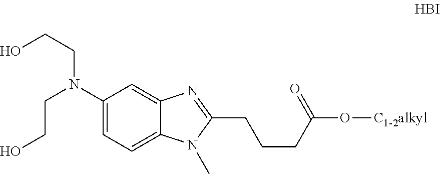
with thionyl chloride in the presence of an organic solvent at about 25° C. for at least about 20 hours to form a first product solution;
(b) combining the first product solution with an aqueous solution of hydrochloric acid to form a biphasic mixture;
(c) removing the organic phase from the biphasic mixture to provide a first aqueous mixture;
(d) heating the first aqueous mixture for 4 to 5 hours at 85° C. to 90° C.;
(e) distilling the first aqueous mixture at between 50° C. to 60° C. to remove between 65-75% of the aqueous solution of hydrochloric acid from the first aqueous mixture to provide a second aqueous mixture;
(f) crystallizing a first portion of bendamustine hydrochloride from the second aqueous mixture;
(g) isolating the first portion of bendamustine hydrochloride from the second aqueous mixture;
(h) heating the first portion of bendamustine hydrochloride in acetone to provide a second portion of bendamustine hydrochloride; and
(i) isolating the second portion of bendamustine hydrochloride;
wherein the method does not include a step comprising recrystallization of bendamustine hydrochloride using ethanol.

2. The method of claim 1, wherein the organic solvent is chloroform.

3. The method of claim 1, wherein contacting step (a) is performed for 20 to 24 hours.

4. The method of claim 1, wherein the aqueous solution of hydrochloric acid is about 32% hydrochloric acid in water.

5. The method of claim 1, wherein charcoal is added to the first aqueous mixture prior to heating step (d).

6. The method of claim 5, wherein the charcoal is filtered from the first aqueous mixture prior to distilling step (e).

7. The method of claim 1, wherein distilling step (e) is performed under reduced pressure.

8. The method of claim 1, wherein heating step (h) is performed for at least one hour.

9. The method of claim 1, wherein heating step (h) is performed at least three consecutive times.

10. The method of claim 1, wherein heating step (h) is performed four consecutive times.

11. The method of claim 1, wherein each of the isolation steps is via filtration.

12. The method of claim 1 further comprising the following steps:
(j) dissolving the second portion of bendamustine hydrochloride in an aqueous solution of hydrochloric acid to form a third aqueous mixture;
(k) heating the third aqueous mixture at 85° C. to 90° C. for 4 to 5 hours;
(l) distilling the third aqueous mixture at between 50° C. to 60° C. to remove up to half of the solvent to provide a fourth aqueous mixture;
(m) crystallizing a third portion of bendamustine hydrochloride from the fourth aqueous mixture;
(n) isolating the third portion of bendamustine hydrochloride from the fourth aqueous mixture;
(o) heating the third portion of bendamustine hydrochloride in acetone to provide a fourth portion of bendamustine hydrochloride; and
(p) isolating the fourth portion of bendamustine hydrochloride.

13. The method of claim 12, wherein dissolving step (j) further includes adding charcoal to the third aqueous mixture.

14. The method of claim 13, wherein the charcoal is filtered from the third aqueous mixture prior to distilling step (l).

15. The method of claim 12, wherein each of the isolation steps is via filtration.

16. A method of removing impurities from bendamustine hydrochloride comprising:
(a) heating bendamustine hydrochloride in an aqueous solution of hydrochloric acid to form a mixture;
(b) distilling off at least part of the aqueous solution of hydrochloric acid from the mixture;
(c) adding water to form a bendamustine solution;
(d) crystallizing bendamustine hydrochloride from the bendamustine solution;
(e) isolating the bendamustine hydrochloride from the bendamustine solution;
(f) heating the isolated bendamustine hydrochloride in acetone.

17. The method of claim 16, wherein heating step (a) is performed at 85° C. to 90° C.

18. The method of claim 16, wherein heating step (a) is performed for at least 4 to 5 hours.

19. The method of claim 16, wherein distilling step (b) is performed at between about 50° C. to 60° C.

20. The method of claim 16, wherein distilling step (b) is performed under reduced pressure.

21. The method of claim 16, wherein heating step (f) is performed at reflux temperature.

22. The method of claim 16, wherein heating step (f) is performed for at least one hour.

23. The method of claim 16, wherein heating step (f) is performed at least three times.

24. The method of claim 16, wherein heating step (f) is performed four times.

25. The method of claim 16, wherein further comprising adding active charcoal to the mixture of heating step (a).

26. The method of claim 25, further comprising filtering off the active charcoal prior to distillation step (b).

27. The method of claim 16, wherein the isolation is via filtration.
